(19) United States
(12) Patent Application Publication
(10) Pub. No.: US 2026/0071366 A1
(43) Pub. Date: Mar. 12, 2026

(54) METHOD FOR PREPARING MYCELIUM SHEET OR PANEL MATERIAL FOR FURTHER PROCESSING, AND MYCELIUM SHEET OR PANEL MATERIAL PRODUCED THEREBY

(71) Applicant: Ecovative LLC, Green Island, NY (US)

(72) Inventors: Alex Stephen Friedman, Troy, NY (US); Peter James Mueller, Poestenkill, NY (US)

(21) Appl. No.: 19/325,920

(22) Filed: Sep. 11, 2025

Related U.S. Application Data
(60) Provisional application No. 63/694,038, filed on Sep. 12, 2024.

(51) Int. Cl. D04H 3/015; C12N 1/14; C12R 1/645; D04H 3/10

(52) U.S. Cl. CPC D04H 3/015; C12N 1/145; D04H 3/10; C12R 2001/645; D10B 2401/06; D10B 2401/14

(57) ABSTRACT

A mycelium sheet or panel material which has had an upper portion of its growing surface removed prior to or concurrently with harvest, provides a more homogeneous surface for later processing of the mycelium sheet or panel material into a mycelium-based textile. An additional rinsing step of a mycelium sheet or panel material which has had an upper portion removed provides a surface which is even more amenable to textile processing into a mycelium-based textile. Such removal and rinsing steps produce a mycelium-based sheet or panel material which provide more homogeneity, such as in color appearance or texture, thereby leading to a more homogeneous mycelium-based textile end product. By addressing and eventually removing fungal growth metabolites during the mycelial growth and harvest processes, later textile processing challenges with the mycelium-based material may be eliminated. Undesirable surface irregularities in the mycelium-based material of final textile products may also be eliminated.

### CROSS-REFERENCE TO RELATED APPLICATION

The present application claims filing benefit of U.S. Provisional Patent Application No. 63/694,038 having a filing date of Sep. 12, 2024, which is hereby incorporated by reference in its entirety.

### BACKGROUND OF THE DISCLOSURE

### Field

The present application relates to methods for processing mycelium sheets and mycelium sheet materials produced thereby.

### Description

Mycelium-based materials have demonstrated significant promise in their potential for commercialization as alternatives to traditional textile materials, such as animal-based leather, and petroleum-based sheeting (such as foams), which leather and petroleum-based products present increasing challenges with respect to environmental sustainability (and associated philosophical issues). The future availability of seemingly endless amounts of animal hides, let alone inexpensive animal hides, has now been called into question, given the level of natural resources required to support manufacture of such materials and the potentially detrimental chemicals used to support such manufacture. Changes in consumer mindsets for products incorporating such materials have also added a further dimension to the uncertain future of traditional textile materials.

In contrast, mycelium-based materials have demonstrated the ability to perform sought-after functionality, with desirable environmentally-friendly and/or sustainable carbon footprints, especially when compared to certain, currently-practiced, animal-based or petroleum-based material sources. However, these new materials present challenges in their own right, such as the need to provide equivalent and consumer-expected product performance. In particular, such mycelium-based materials have cellular structures and other physical attributes presenting manufacturing challenges that are otherwise not present with animal-based or synthetic polymer-based materials. For example, grown mycelial sheeting often takes on color and textural features, making them on occasion more challenging to work with and to fashion into material more closely resembling traditional textile materials. Further, it may be challenging to produce mycelium-based materials demonstrating expected product strength, integrity, and lifespans similar to traditional textile materials, but without overuse of synthetic or environmentally-sensitive chemistries. Objectives of this disclosure include methods for addressing at least some of these unique manufacturing challenges, without sacrificing material performance.

### SUMMARY OF THE DISCLOSURE

For purposes of summarizing the disclosure and the advantages achieved over the prior art, certain objects and advantages of the disclosure have been described herein. Of course, it is to be understood that not necessarily all such objects or advantages may be achieved in accordance with any particular embodiment of the disclosure. Thus, for example, those skilled in the art will recognize that the disclosure may be embodied or carried out in a manner that achieves or optimizes one advantage or group of advantages as taught herein without necessarily achieving other objects or advantages as may be taught or suggested herein.

In a first aspect of the disclosure, a method for preparing a mycelium sheet or panel material for further processing is disclosed. The method includes the steps of: (a) growing a mycelium sheet or panel material including hyphae in a growth environment and upon a nutritive substrate, with the growing mycelium sheet or panel material including an original upper surface, (b) removing an upper portion of the growing mycelium sheet or panel material, either prior to or in conjunction with a mycelium sheet or panel material harvest, thereby exposing a secondary upper surface on a base layer, which had been located beneath the original upper surface, and (c) processing the growing mycelium sheet or panel material including the secondary upper surface into a mycelium-based textile product.

In a further aspect of the method, the method further includes compressing the growing mycelium sheet or panel material. In still a further aspect of the method, the compressing step is performed by one or more rollers or pressure plates. In yet a further aspect of the method, the method further includes rendering the growing mycelium sheet or panel material inert. Such rendering may be accomplished by exposure of the growing mycelium sheet or panel material to an action comprising (or selected from the group consisting of) heating and/or drying.

In still another aspect of the method, the removing step is accomplished by inserting a perforated layer upon the growing mycelium sheet or panel material, whereby the growing mycelium sheet or panel hyphae penetrate the perforated layer to create the upper portion of the growing mycelium sheet or panel material, and the perforated layer, along with mycelium hyphae which have subsequently grown through the perforated layer are eventually removed together, thereby exposing a secondary upper surface of growing mycelium which had most recently not been the mycelium sheet or panel material original upper surface. Essentially, the perforated layer becomes embedded in the growing mycelium sheet or panel material and then is used to delaminate that portion of the growing mycelium located above it (the upper portion of the growing mycelium sheet or panel) from the remaining base layer of the growing mycelium sheet or panel material.

In still another aspect of the method, the upper portion removing step is accomplished by a cutting mechanism, such as for example a bandsaw or a wire cutter. The bandsaw may be a horizontal bandsaw.

In yet another aspect of the method, the method further includes a rinsing step following the removing step. The rinsing step itself serves as a secondary removing step for removing secondary metabolites that were not removed by the physical removal step (of cutting or delaminating) which effectively removed the bulk of primary metabolites situated at the youngest growing hyphal tips.

In yet another aspect of the method, the method further includes a harvesting step, wherein the mycelium sheet or panel material is removed from the nutritive substrate. In still another aspect of the method, a rinsing step follows the harvesting step.

In yet a further aspect of the disclosure, a method for preparing a mycelium sheet or panel material for further textile processing is disclosed. The method includes the steps of: (a) growing a mycelium sheet or panel material including hyphae in a growth environment and upon a nutritive substrate, with the growing mycelium sheet or panel material including an original upper surface, (b) removing an upper portion of the growing mycelium sheet or panel material, thereby exposing a secondary upper surface on a base layer of the mycelium sheet or panel material, which secondary upper surface had been located beneath the original upper surface, and further, which base layer of the mycelium sheet or panel material still being situated upon the nutritive substrate (which removing action may be accomplished by a cutting or delaminating mechanism), (c) harvesting the mycelium sheet or panel material from the nutritive substrate (which harvesting may be accomplished by various mechanisms, such as by cutting using a blade or saw or delaminating using an embedded perforated layer), and (d) processing the harvested growing mycelium sheet or panel material including the secondary upper surface into a mycelium-based textile product.

In a further aspect of the method, the mycelium sheet or panel material includes aerial mycelium, which originated from extra-particle aerial mycelial growth off of the nutritive substrate. In yet a further aspect of the method, the nutritive substrate is selected from the group consisting of a liquid state nutritive substrate, a solid-state nutritive substrate, or a combination thereof. For instance, the nutritive substrate may be a liquid state nutritive substrate in one aspect. In other aspects, the nutritive substrate may be a solid-state nutritive substrate. In still another aspect of the method, the method further includes rinsing the harvested mycelium sheet or panel material.

In another aspect of the disclosure, a mycelium sheet or panel material produced by any of the described claimed methods is also contemplated. In some aspects, the mycelium sheet or panel material may demonstrate a noticeable reduction in color than a comparable mycelium sheet or panel material not having an upper portion removed prior to further processing. In some aspects, the mycelium sheet or panel material may demonstrate a noticeable reduction in surface irregularities than a comparable mycelium sheet or panel material not having an upper portion removed prior to further processing. In some aspects, the mycelium sheet or panel material may demonstrate a noticeable reduction in brittleness than a comparable mycelium sheet or panel material not having an upper portion removed prior to processing.

All of these embodiments are intended to be within the scope of the disclosure herein disclosed. These and other embodiments of the present disclosure will become readily apparent to those skilled in the art from the following detailed description of the alternative embodiments having reference to the attached figures, the disclosure not being limited to any particular alternative embodiment(s) disclosed.

### BRIEF DESCRIPTION OF THE FIGURES

FIG. 1 is a partial cross-sectional view of a grown aerial mycelial sheet/panel on a solid particulate nutritive substrate in accordance with one embodiment of the disclosure.

FIG. 2 is a partial cross-sectional view of the grown aerial mycelial sheet/panel of FIG. 1 being removed from the solid particulate nutritive substrate in accordance with one embodiment of the disclosure.

FIG. 3 is a partial cross-sectional view of a grown aerial mycelial sheet/panel on a solid particulate nutritive substrate in accordance with one embodiment of the disclosure.

FIG. 4 is a partial cross-sectional view of the grown aerial mycelial sheet/panel of FIG. 3 being removed from the solid particulate nutritive substrate in accordance with one embodiment of the disclosure.

FIG. 5 is a process flow diagram illustrating a series of method steps in accordance with several embodiments of the disclosure.

FIG. 6 is a series of photographic images illustrating removal of a top layer of mycelium (and in particular aerial mycelium) from a mycelium panel via a net (as seen in the left most images). The difference between a panel with the top section intact and the top section removed, following a drying step is shown in the rightmost image.

FIG. 7 is a series of photographic images with a top section of an aerial mycelium panel removed comparing the results of various secondary processing steps (or processing steps) and metabolite-rinsing treatments with subsequent compression and drying steps.

FIG. 8 illustrates a process flow diagram for a method for top section of mycelium panel removal, and supplemental metabolite rinsing steps.

### DETAILED DESCRIPTION

The following discussion presents detailed descriptions of several embodiments of methods for preparing mycelium sheets or panel materials for further processing, such as for preparing mycelium sheets or panel materials for later physical and/or chemical treatments to produce textile-like materials. In accordance with the disclosure, mycelium sheet or panel materials are grown in appropriate growth environments (as described below), and include one of various process steps for removal or eventual removal, of an upper portion or growth layer of a grown mycelium sheet or panel material, in preparation for further treatments. This removal of upper portion results in the removal of primary metabolites, which are theorized to lead to color and texture variations in the normally exposed mycelium sheet or panel material surface. It has been found that the removal of an upper portion of a grown mycelium sheet or panel material (and in one embodiment, an aerial mycelium sheet or panel material) imparts color and texture improvement benefits (in terms of elimination in color and texture variations) to the mycelium sheet or panel material, which translate into improvements in a final mycelium-based textile product produced therefrom. Such removal of an uppermost layer improves irregular coloration and other physical attributes of a textile product fashioned from the mycelium-based sheet or panel material. By removing the upper portion, a lower portion (that is a portion lower in elevation with respect to distance from the nutritive substrate) is thereby exposed (and becomes the new upper surface). Additionally, an optional, later rinsing step of the altered grown material (“altered” being the grown material which has had its upper surface removed (that is the portion of the mycelium sheet or panel most distanced from the nutritive substrate)), results in removal of secondary metabolites away from the remaining (unremoved) material. Such removal of secondary metabolites is also theorized to reduce or prevent at least discoloration and brittleness in a final mycelium-based textile product (in one embodiment aerial mycelium-based textile product) compared to such a product that has not undergone such removal (such as lifting off or cutting) and rinse steps. Such removal of an upper surface as described may also result in a smoother mycelium-based end product, having less heterogeneous surface configurations.

It is theorized that the uppermost layer of a growing mycelium sheet or panel material (panel being a relatively thick mycelium planar structure) contains most of the metabolites within the mycelium tissue that are responsible for surface defects, such as discoloration and other textural irregularities (especially once the mycelium, such as aerial mycelium has been compressed and rendered inert (such as by heating/drying)). By physical removal of an uppermost portion of a mycelium sheet or panel material, the primary metabolites are physically removed. Of course, removal of only that upper portion of a mycelium sheet or panel material (such as an aerial mycelium sheet or panel material) which is necessary to eliminate the highest concentration of primary metabolites is most desirable, such that as much of the remainder mycelial tissue may be preserved for later use in a textile product. By then rinsing off the remaining mycelial tissue (that is that larger portion of the mycelial tissue which was not removed and either discarded or used for some other purpose, such as for example a source of a second flush of mycelial growth), secondary metabolites still present near the newly exposed outer surface of the remainder mycelial tissue, can be removed.

The various steps involved in primary and secondary metabolite removal are described below, following a general description of the growth conditions for mycelial tissue, and in particular, extra-particle aerial mycelial tissue in accordance with the disclosure (which will ultimately lead to the harvest of mycelium (and in particular aerial mycelium)).

### Definitions

The mycelia of the present disclosure are growth products obtained from a growth matrix (including nutritive substrate) incubated for a period of time (i.e., an incubation time period) in or on a substrate-supporting surface of a support structure (or tool) in a growth environment, as disclosed herein. For the purposes of this disclosure the following terms are given their respective meanings.

“Mycelium” as used herein refers to a connective network of fungal hyphae, with mycelia being the plural form of mycelium.

“Hyphae” as used herein refers to branched filament vegetative cellular structures that are interwoven to form mycelium.

“Substrate” or “Nutritive substrate” as used herein refers to a material or surface thereof, from or on which an organism lives, grows, and/or obtains its nourishment. In some embodiments, a substrate provides sufficient nutrition to the organism under target growth conditions such that the organism can live and grow without providing the organism a further source of nutrients. A variety of substrates are suitable to support the growth of an aerial mycelium of the present disclosure. Suitable substrates are disclosed, for example, in U.S. Patent Application Publication US2020/0239830A1 to O'Brien et al, the entire contents of which are hereby incorporated by reference in their entirety, to the extent not inconsistent with the content of this disclosure. In some embodiments, the substrate is a natural substrate. Non-limiting examples of a natural substrate include a lignocellulosic substrate, a cellulosic substrate, or a lignin-free substrate. A natural substrate can be an agricultural waste product or one that is purposefully harvested for the intended purpose of food production, including mycelial-based food production. Further non-limiting examples of nutritive substrates suitable for supporting the growth of mycelia of the present disclosure include soy-based materials, oak-based materials, maple-based materials, corn-based materials, seed-based materials and the like, or combinations thereof. The materials can have a variety of particle sizes, as disclosed in US2020/0239830A1, and occur in a variety of forms, including shavings, pellets, chips, flakes, or flour, or can be in monolithic form. Non-limiting examples of suitable substrates for the production of mycelia of the present disclosure include corn stover, maple flour, maple flake, maple chips, soy flour, chickpea flour, millet seed flour, oak pellets, soybean hull pellets and combinations thereof. Additional useful substrates for the growth of mycelia are disclosed herein, but may also include liquid state nutritive substrates as are known in the art.

“Growth media” or “growth medium” as used herein refers to a matrix containing a nutritive substrate and an optional further source of nutrition that is the same or different than the nutritive substrate, wherein the nutritive substrate, the nutrition source, or both are intended for fungal consumption to support mycelial growth.

“Growth matrix” as used herein refers to a matrix containing a growth medium and a fungus. In some embodiments, the fungus is provided as a fungal inoculum; thus, in such embodiments, the growth matrix comprises a fungal-inoculated growth medium. In other embodiments, the growth matrix comprises a colonized substrate.

“Inoculated substrate” as used herein refers to a substrate (or nutritive substrate) that has been inoculated with fungal inoculum. For example, an inoculated substrate can be formed by combining an uninoculated substrate with a fungal inoculum. An inoculated substrate can be formed by combining an uninoculated substrate with a previously inoculated substrate. An inoculated substrate can be formed by combining an inoculated substrate with a colonized substrate.

“Colonized substrate” as used herein refers to an inoculated substrate that has been incubated for sufficient time to allow for fungal colonization. A colonized substrate of the present disclosure can be characterized as a contiguous hyphal mass grown throughout the entirety of the volume of the growth media substrate. The colonized substrate may further contain residual nutrition that has not been consumed by the colonizing fungus. As is understood by persons of ordinary skill in the art, a colonized substrate has undergone primary myceliation, sometimes referred to by skilled artisans as having undergone a “mycelium run.” Thus, in some particular aspects, a colonized substrate consists essentially of a substrate and a colonizing fungus in a primary myceliation phase. For many fungal species, asexual sporulation occurs as part of normal vegetative growth, and as such could occur during the colonization process. Accordingly, in some embodiments, a colonized substrate of the present disclosure may also contain asexual spores (conidia). In some aspects, a colonized substrate of the present disclosure can exclude growth progression into sexual reproduction and/or vegetative foraging. Sexual reproduction includes fruiting body formation (e.g., primordiation and differentiation) and sexual sporulation (meiotic sporulation). Vegetative foraging includes any mycelial growth away from the colonizing substrate (such as aerial growth). Thus, in some further aspects, a colonized substrate can exclude mycelium that is in a vertical expansion phase of growth. A colonized substrate can enter a mycelial vertical expansion phase during incubation in a growth environment of the present disclosure. For example, a colonized substrate can enter a mycelial vertical expansion phase upon introducing aqueous mist into the growth environment and/or depositing aqueous mist onto colonized substrate and/or any ensuing extra-particle growth. In some embodiments, the use of aqueous mist can be adjusted, for example, to desired levels and timing, to affect the topology, morphology, density, and/or volume of the growth.

Any suitable substrate or nutritive substrate can be used alone, or optionally combined with a nutrient source, as media to support mycelial growth. In the case of solid-state platforms (not having a continuous liquid phase throughout portions), the growth media can be hydrated to a final target moisture content prior to inoculation with a fungal inoculum. In a non-limiting example, the substrate or growth media can be hydrated to a final moisture content of at least about 50% (w/w), at most about 95% w/w, within a range of about 50% to about 95%, about 50% to about 90%, about 50% to 85%, about 50% (w/w) to about 80% (w/w), about 50% (w/w) to about 75% (w/w), within a range of about 50% (w/w) to about 65% (w/w), within a range of about 50% (w/w) to about 60% (w/w), or within a range of about 60% (w/w) to about 70% (w/w). Growth media hydration can be achieved via the addition of any suitable source of moisture. In a non-limiting example, the moisture source can be airborne or non-airborne liquid phase water (or other liquids), an aqueous solution containing one or more additives (including but not limited to a nutrient source), and/or gas phase water (or other compound). In some embodiments, at least a portion of the moisture is derived from steam utilized during bioburden reduction of the growth media. In some embodiments, inoculation of the growth media with the fungal inoculum can include a further hydration step to achieve a target moisture content, which can be the same or different than the moisture content of the growth media. For example, if growth media loses moisture during fungal inoculation, the fungal inoculated growth media can be hydrated to compensate for the lost moisture. Liquid state nutritive substrates may also be used in certain embodiments of the disclosure, as further described.

Methods for the production of extra-particle aerial mycelium sheets and panels (and ultimately, aerial mycelium) disclosed herein can include an inoculation stage, wherein an inoculum is used to transport an organism into a nutritive substrate. The inoculum, which carries a desired fungal strain, is produced in sufficient quantities to inoculate a target quantity of nutritive substrate. The inoculation can provide a plurality of myceliation sites (nucleation points) distributed throughout the nutritive substrate. Inoculum can take the form of a liquid, a slurry, or a solid, or any other known vehicle for transporting an organism from one growth-supporting environment to another. Generally, the inoculum comprises water, carbohydrates, sugars, vitamins, other nutrients, and fungi. The inoculum may contain enzymatically available carbon and nitrogen sources (e.g., lignocellulosic biomass, chitinous biomass, carbohydrates) augmented with additional micronutrients (e.g., vitamins, minerals). The inoculum can contain inert materials (e.g., perlite). In a non-limiting example, the fungal inoculum can be a seed-supported fungal inoculum, a feed-grain-supported fungal inoculum, a seed-sawdust mixture fungal inoculum, or another commercially available fungal inoculum, including specialty proprietary spawn types provided by inoculum retailers. In some aspects, a fungal inoculum can be characterized by its density. In some embodiments, a fungal inoculum has a density of about 0.1 gram per cubic inch to about 10 grams per cubic inch, or from about 1 gram per cubic inch to about 7 grams per cubic inch. A skilled person can modify variables including the nutritive substrate or growth media component identities, nutritive substrate or growth media nutrition profile, nutritive substrate or growth media moisture content, nutritive substrate or growth media bioburden, inoculation rate, and inoculum constituent concentrations to arrive at a suitable medium to support mycelial and in some instances extra-particle aerial mycelial growth. In some embodiments, the inoculation rate can be expressed as a percentage of the target volume of the substrate or growth media (% (v/v)). In some embodiments, the inoculation rate can range from about 0.1% (v/v) to about 80% (v/v). In some embodiments, the inoculation rate is at most about 50% (v/v), at most about 45% (v/v), at most about 40% (v/v), at most about 30% (v/v), at most about 25% (v/v), at most about 20% (v/v), at most about 15% (v/v), at most about 10% (v/v) or at most about 5% (v/v). In some embodiments, the inoculation rate is about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 11% (v/v), about 12% (v/v), about 13% (v/v), about 14% (v/v), about 15% (v/v), about 16% (v/v), about 17% (v/v), about 18% (v/v), about 19% (v/v), about 20% (v/v), about 21% (v/v), about 22% (v/v), about 23% (v/v), about 24% (v/v), about 25% (v/v), about 26% (v/v), about 27% (v/v), about 28% (v/v), about 29% (v/v) or about 30% (v/v); or any range therebetween. In some embodiments, the inoculation rate can be expressed as a percentage of the target dry mass of the nutritive substrate or growth media (% (w/w)). In some embodiments, the inoculation rate can range from about 0.1% (w/w) to about 80% (w/w). In some embodiments, the inoculation rate is at most about 50% (w/w), at most about 45% (w/w), at most about 40% (w/w), at most about 30% (w/w), at most about 25% (w/w), at most about 20% (w/w), at most about 15% (w/w), at most about 10% (w/w) or at most about 5% (w/w). In some embodiments, the inoculation rate is about 1% (w/w), about 2% (w/w), about 3% (w/), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w) or about 30% (w/w); or any range therebetween.

“Aerial mycelium” as used herein refers to mycelium obtained from extra-particle aerial mycelial growth, and which is substantially free of growth matrix (i.e. solid particulate material).

“Extra-particle mycelial growth” (EPM) as used herein refers to mycelial growth from a nutritive substrate particle, which can be characterized in some instances, as being “aerial”.

“Extra-particle aerial mycelial growth” or “extra-particle aerial mycelium” as used herein refers to a distinct mycelial growth that occurs away from and outward from the surface of a growth matrix (including nutritive substrate having solid particulate material). It has not yet been physically removed from the typically underlying, growth matrix (including particulate nutritive substrate), but has grown so that it extends away from the growth matrix, as opposed to merely between portions of growth matrix. In some embodiments, it extends in a generally vertical orientation, perpendicular to the growth matrix (including nutritive substrate) surface, situated in or upon the support structure, substrate-supporting surface. Extra-particle aerial mycelial growth can therefore exhibit negative gravitropism, in that it grows in a direction opposite that of the direction of gravity. In a geometrically unrestricted scenario, extra-particle aerial mycelial growth could be described as being negatively gravitropic, positively gravitropic, or neutrally gravitropic, aerial, and radial in which growth will expand in all directions from its point source.

“Positive gravitropism” (or positively gravitropic) as used herein refers to growth that preferentially occurs in the direction of gravity.

“Negative gravitropism” as used herein refers to mycelial growth that preferentially occurs in the direction away from the direction of gravity. As disclosed herein, extra-particle aerial mycelial growth can exhibit negative gravitropism. Without being bound by any particular theory, this may be attributable at least in part to the geometric restriction of the growth format, wherein an uncovered tool having at least a substrate-supporting surface, supports or contains a growth matrix (including nutritive substrate). With such geometric restriction, growth will primarily occur along the unrestricted dimension(s), which in the scenario is primarily vertically (negatively gravitropic) if the tool is positioned such that its surface or opening (in the case of a four solid-walled open container or flat surfaced tool) is facing vertically upward in orientation (and the fungal organism (e.g. hyphae of mycelium) is attracted to airborne mist).

Aerial mycelia (based on extra-particle aerial mycelial growth) of the present disclosure can be grown in a matter of weeks or days. This feature is of practical value, at least in the production of food ingredients or food products, where time and efficiency are at a premium. Accordingly, the presently disclosed method of making an aerial mycelium (off of extra-particle aerial mycelial growth) comprises incubating a growth matrix (including nutritive substrate) in a growth environment for an incubation time period of up to about 3 weeks. In some embodiments, the incubation time period can be within a range of about 4 days to about 17 days. In some further embodiments, the incubation time period can be within a range of about 7 days to about 16 days, within a range of about 8 days to about 15 days, within a range of about 9 days to about 15 days, within a range of about 9 days to about 14 days, within a range of about 8 to about 14 days, within a range of about 7 days to about 13 days, or within a range of about 7 days to about 10 days. In some more particular embodiments, the incubation time period can be about 7 days, about 8 days, about 9 days, about 10 days, about 11 days, about 12 days, about 13 days, about 14 days, about 15 days or about 16 days, or any range therebetween.

Advantageously, incubating a growth matrix comprising a colonized nutritive substrate (wherein the colonized nutritive substrate comprises a growth medium previously colonized with mycelium of a fungus) in a growth environment of the present disclosure can result in earlier expression of extra-particle aerial mycelial tissue compared to incubation of a growth matrix (including nutritive substrate) comprising substantially the same or a similar growth medium and a fungal inoculum, wherein the fungal inoculum contains a fungus. Accordingly, a method of making an aerial mycelium (based on extra-particle aerial mycelial growth) of the present disclosure can comprise incubating a growth matrix comprising a colonized nutritive substrate (wherein the colonized nutritive substrate comprises a growth medium previously colonized with mycelium of a fungus) in a growth environment for an incubation time period, and producing extra-particle aerial mycelial growth therefrom, wherein the incubation time period is at least about 1 day, at least about 2 days, at least about 3 days, or at least about 4 days less than the incubation time period for producing extra-particle aerial mycelial growth from a growth matrix comprising a growth medium and a fungal inoculum, wherein the fungal inoculum comprises a fungus.

In some other embodiments, the incubation time period ends no later than when a visible fruiting body forms. In a non-limiting example, the incubation time period can end prior to a karyogamy or meiosis phase of the fungal reproductive cycle. In some other embodiments, the incubation time period ends when a visible fruiting body forms. As disclosed herein, aerial mycelia (based on the extra-particle aerial mycelial growth) of the present disclosure can be prepared without the formation of a visible fruiting body, thus, in some embodiments, an incubation time period can end without regard to the formation of a visible fruiting body. Trial incubation runs can be used to inform the period of time in the growth environment during which sufficient extra-particle aerial mycelial growth product occurs (e.g., aerial mycelial growth of a predetermined thickness) without the formation of visible fruiting bodies.

### Additional Definitions and Methods Related to Growth Environment

U.S. Patent Application Publication 2015/0033620 to Greetham et al., the entire contents of which is hereby incorporated by reference in its entirety to the extent not inconsistent with the content of this disclosure, describes techniques for growing a material comprising aerial mycelium, referred to in that application as a “mycological biopolymer.” As described therein, a mycological biopolymer product provided by that disclosed method is characterized as containing a homogenous biopolymer matrix that is comprised predominantly of fungal chitin and trace residues (e.g., beta-glucan, proteins). The mycological biopolymer is up-cycled from domestic agricultural lignocellulosic waste and is made by inoculating the substrate made of domestic agricultural lignocellulosic waste with a selected fungus in a container that is sealed off from the ambient environment external to the container. In addition to the substrate and fungal inoculum, the container contains a void space. A network of undifferentiated aerial mycelium comprising a chitin-polymer grows into and fills the void space of the container. The chitin-polymer-based aerial mycelium is subsequently extracted from the substrate and dried. As further described in US2015/0033620, the environmental conditions for producing the mycological biopolymer product described therein, i.e., a high carbon dioxide (CO₂) content (about 3% to about 7% by volume) and an elevated temperature (from about 85° F. to about 95° F.), prevent full differentiation of the fungus into a mushroom, as evidenced by the absence of a visible fruiting body.

In one aspect, the present disclosure provides a mycelium, alternatively, extra-particle aerial mycelium (and subsequently harvested aerial mycelium) grown using the described tool apparatus, methods, and systems incorporating the same. In a further aspect, the aerial mycelium (based on extra-particle aerial mycelium) does not contain a visible fruiting body.

As described in International Patent Publication WO2019/099474A1 to Winiski et al., the entire contents of which is hereby incorporated by reference in its entirety to the extent not inconsistent with the content of this disclosure, another method of growing a mycological biopolymer material employs incubation of a substrate with nutritive value inoculated with a fungus in containers that are placed in a closed incubation chamber with air flows passed over each container while the chamber is maintained with a predetermined environment of humidity, temperature, carbon dioxide, and oxygen.

The aerial mycelia (from extra-particle aerial mycelial growth) of the present disclosure are growth products obtained from an inoculated nutritive substrate incubated for a period of time (i.e., an incubation time period) in a growth environment, as disclosed herein.

In some aspects, a method of making an aerial mycelium (from extra-particle aerial mycelial growth) of the present disclosure comprises placing a growth matrix (containing nutritive substrate) in contact with a tool in the described growth environment. In some aspects, the tool can have a substrate-supporting surface having a surface area. In some embodiments, the surface area can be at least about 1 square inch. In some embodiments, the surface area can be at most about 2000 square feet. In some embodiments, the growth matrix (including nutritive substrate) can be placed in contact with the substrate-supporting surface, e.g., placed directly or indirectly on top of or distributed across the substrate-supporting surface. In some embodiments, the substrate-supporting surface can be a planar surface, or a non-planar surface (as further illustrated below with integrally formed physical spacers). Non-limiting examples of a tool include a tray, a sheet, a screen, a pan or table, a conveyer belt, a net or a web. In some embodiments, the tool can have at least one side wall and a floor. In other embodiments, the at least one side wall and floor can be solid. In still further embodiments, the tool can have four side walls. In another embodiment, the one or more side walls can be porous, perforated, or otherwise open. In some embodiments, the substrate-supporting surface (such as a floor) and the at least one side wall can together form a cavity. In other embodiments, the support structure may itself include one or more recesses that form one or more cavities (which cavities include one or more substrate-supporting surfaces). In other embodiments, the support structure may itself include one or more recesses and also includes at least one side wall. In some embodiments, the growth matrix can be placed or packed in the tool cavity or cavities. In some embodiments, the tool can be an uncovered tool. In some other embodiments, the tool can have a lid, the lid having at least one opening, or the tool can be covered at least in part with a perforated barrier. Non-limiting embodiments of a tool having a lid with an opening are disclosed in US2015/0033620A1. An uncovered tool, or a tool having a lid with an opening or a perforated barrier, and further having growth matrix (including nutritive substrate) on or within the tool, can allow for aqueous mist to be deposited onto the growth matrix (and nutritive substrate) surface, and/or onto any resulting mycelial growth that may be occurring.

In some embodiments, the tool may include a perforated material, such as a net, scrim-like material, screen, or mesh situated immediately above the growth matrix, through which mycelial hyphae may grow away from the nutritive substrate. The perforated material, in one embodiment, includes perforations sized to allow for the easy passage of hyphae away from the growth matrix. Such perforated material is in one embodiment formed from a material that is chemically or biologically inert with respect to the growing mycelium. That is, such perforated material does not have any deleterious effects on the growing mycelium. Such perforated material may, in one embodiment, be fashioned from polymeric, metallic, glass, organic, or ceramic substances. For instance such perforated material, may in one embodiment be formed from a nonwoven material or woven web, such as for instance, from a perforated film, a fibrous nonwoven material (such as a spunbond or meltblown web or a paper-like cellulosic material), alternatively from a fibrous woven material such as a cotton scrim, or similar material. Alternatively, such a perforated material may be formed from a metallic screen or such. Such perforated material may, in one embodiment, remain with the growth matrix upon removal of the extra-particle aerial mycelium. In an alternative embodiment, such perforated material may be removed along with the extra-particle aerial mycelium upon harvest, such that it provides some advantageous attribute to the removed extra-particle, aerial mycelium (and harvested aerial mycelium itself). For instance, such included perforated material may provide additional strength to the grown mycelium sheet or panel material.

In a further embodiment, at some beneficial point in the growth cycle of the extra-particle aerial mycelium, a secondary perforated material (such as those previously described), may be placed upon the current upper surface of the growing mycelium. The growing mycelium may then be permitted to continue its growth through the secondary perforated material. Such secondary perforated material may then be used to remove an upper portion of the final grown mycelium sheet or panel material, in order to provide multiple attributes to the grown extra-particle aerial mycelium (and resulting aerial mycelium). Such attributes may include one or more of at least: a reduction in brittleness of the final product, a reduction in color variation or other textural variation in the final product (at least along the surface from which the secondary perforated material was removed), and simplified later processing of the grown mycelium sheet or panel material.

“Growth environment” as used herein refers to an environment that supports the growth of mycelia, as would be readily understood by a person of ordinary skill in the art in the mycelial cultivation industry, and which contains a growth atmosphere having a gaseous environment of carbon dioxide (CO₂), oxygen (O₂), and a balance of other atmospheric gases including nitrogen (N₂), and is further characterized as having a relative humidity. In some aspects of the present disclosure, the growth atmosphere can have a CO₂ content of at least about 0.02% (v/v), at least about 5% (v/v), less than about 8% (v/v), less than about 10% (v/v), from about 0.02% and 10%, from about 0.02% and 8%, from about 5% and 10%, or from about 5% and 8%. In some other aspects, the growth atmosphere can have an O₂ content of at least about 12% (v/v), or at least about 14% (v/v), and at most about 21% (v/v). In yet other aspects, the growth atmosphere can have an N₂ content of at most about 79% (v/v). Each foregoing CO₂, O₂, or N₂ content is based on a dry gaseous environment, notwithstanding the growth environment atmosphere relative humidity.

In some further aspects, a method of making an aerial mycelium (from extra-particle aerial mycelial growth) of the present disclosure comprises incubating the growth matrix (including nutritive substrate) in a growth environment, wherein the growth environment has a temperature that supports mycelial growth. In some embodiments, the growth environment has a temperature within a range of about 55° F. to about 100° F., or within a range of about 60° F. to about 95° F. In some more particular embodiments, the growth environment has a temperature within a range of about 80° F. to about 95° F., or within a range of about 85° F. to about 90° F. throughout the incubation time period. In other embodiments, the growth environment has a temperature within a range of about 60° F. to about 75° F., within a range of about 65° F. to about 75° F., or within a range of about 65° F. to about 70° F. In some embodiments, the growth environment temperature can be tuned to optimize for the growth of a particular fungal genus, species, or strain.

In some aspects of the present disclosure, the growth environment suitable for the growth of the aerial mycelia (from extra-particle aerial mycelial growth) of the present disclosure can be a dark environment. “Dark environment” as used herein in connection with a growth environment would be readily understood by a person of ordinary skill in the art in the mycelial cultivation industry and refers to an environment without natural or ambient light, and without growing lights.

Exposing fungi to white light, and especially blue light, has been associated with the induction of fruiting and the enhancement of production efficiency of oyster mushrooms (e.g., see Roshita & Goh, AIP Conference Proceedings 2030, 020110 (2018)), the entire contents of which are hereby incorporated by reference in their entirety to the extent not inconsistent with the content of this disclosure. An aerial mycelium for some genus of the present disclosure, such as Ganoderma, absent visible fruiting bodies, can be prepared by the methods of the present disclosure in the presence of white light, which includes blue light. Aerial mycelium (from extra-particle aerial mycelial growth) prepared in the presence of white light was consistent in yield, thickness, density, morphology and in the absence of visible fruiting bodies when compared to control aerial mycelia produced under the same growth conditions but in a dark environment. Thus, in some embodiments, a growth environment suitable for the growth of the aerial mycelia (from extra-particle aerial mycelial growth) of the present disclosure is not a dark environment. In some embodiments, the growth environment does not exclude light. In some embodiments, the growth environment can include natural light. In some embodiments, the growth environment can include ambient light. In some embodiments, the growth environment can include a growing light.

As disclosed in US2015/0033620, environmental conditions for producing a mycological biopolymer include a CO₂ content of about 3% to about 7% (v/v) to prevent full differentiation of the fungus into a mushroom. Accordingly, in some aspects, the present disclosure provides for methods of producing an aerial mycelium (from extra-particle aerial mycelial growth) in a growth environment comprising a growth atmosphere, wherein the growth atmosphere can have a CO₂ content within a range of about 3% (v/v) to about 7% (v/v), or within a range of about 5% (v/v) to about 7% (v/v). In some embodiments, the growth atmosphere can have a CO₂ content of about 3%, about 4%, about 5%, about 6%, or about 7% (v/v), or any range therebetween.

Aerial mycelium (from extra-particle aerial mycelial growth) of the present disclosure can be produced without visible fruiting bodies under conditions wherein aqueous mist is introduced into a growth environment having a growth atmosphere containing much lower CO₂ content. For example, it has been found that aerial mycelia (from extra-particle aerial mycelial growth) obtained from a growth environment of circulating mist and an atmosphere having a mean CO₂ content of about 0.04% (v/v) over the course of the incubation time period or having a mean CO₂ content of about 2% (v/v) over the incubation time period were similar in yield, thickness, density, and morphology to aerial mycelia (from extra-particle aerial mycelial growth) obtained via growth in an atmosphere having a mean CO₂ content of 5% (v/v) but otherwise identical growth conditions. Furthermore, aerial mycelia (from extra-particle aerial mycelial growth) of increased thickness can be obtained via incubation in a growth environment described herein and characterized as having particular misting profiles. The present disclosure advantageously provides for methods of making aerial mycelia (from extra-particle aerial mycelial growth) of increased thickness, absent visible fruiting bodies, by adopting preselected misting profiles and employing misting deposition methodologies, without requiring a high CO₂ content growth environment. The ability to increase aerial mycelial thickness (from extra-particle aerial mycelial growth), absent visible fruiting bodies, by tuning mist deposition uniformity and rate can also advantageously reduce incubation time periods, thereby allowing more efficient production of aerial mycelia (from extra-particle aerial mycelial growth) and reduced risk of microbial contamination that can occur in high moisture environments.

Thus, the present disclosure provides for methods of growing aerial mycelia (from extra-particle aerial mycelial growth) in a growth environment comprising a growth atmosphere having markedly reduced CO₂ content and with more uniform deposition of mist and/or control of mist placement, should targeted mist placement be necessary or desirable. Accordingly, in some embodiments, the growth atmosphere CO₂ content can be less than about 3% (v/v). In some embodiments, the growth atmosphere CO₂ content can be no greater than about 2.9% (v/v), no greater than about 2.8% (v/v), no greater than about 2.7% (v/v), no greater than about 2.6% (v/v) or no greater than about 2.5% (v/v). In some further embodiments, the growth atmosphere CO₂ content can be less than 2.5% (v/v). In some embodiments, a growth atmosphere of the present disclosure can have a CO₂ content of at least about 0.02% (v/v). In some embodiments, a growth atmosphere of the present disclosure can have a CO₂ content of at least about 0.03% (v/v). In some further embodiments, the growth atmosphere CO₂ content can approximate ambient atmospheric CO₂ content; for example, the growth atmosphere CO₂ content can be at least about 0.04% (v/v). In some more particular embodiments, the growth atmosphere CO₂ content can be within a range of about 0.02% to about 3% (v/v), about 0.02% to about 2.5% (v/v), about 0.03% to about 3% (v/v), about 0.03% to about 2.5% (v/v), about 0.04% to about 3% (v/v), or about 0.04% to about 2.5% (v/v).

In other embodiments, the growth atmosphere CO₂ content can be within a wider range. Thus, in some embodiments, the growth atmosphere CO₂ content can be within a range of about 0.02% to about 7% (v/v), within a range of about 0.04% to about 7% (v/v), within a range of about 0.1% to about 7% (v/v), within a range of about 0.2% to about 7% (v/v), within a range of about 1% to about 7% (v/v), or within a range of about 2% to about 7% (v/v); or can be within a range of about 0.02% to about 5% (v/v), within a range of about 0.04% to about 5% (v/v), within a range of about 0.1% to about 5% (v/v), within a range of about 0.2% to about 5% (v/v), or within a range of about 1% to about 5% (v/v). In some more particular embodiments, the growth atmosphere CO₂ content can be about 1%, about 2%, about 3%, or any range therebetween. In yet other embodiments, the growth atmosphere CO₂ content can be a mean CO₂ content over the course of the incubation time period. In some embodiments, the growth atmosphere mean CO₂ content can be less than about 3% (v/v), less than 2.5% (v/v), or no greater than about 2% (v/v) over the course of the incubation time period.

It is understood that fungal growth requires respiration, which can increase CO₂ content and decrease oxygen (O₂) content in the growth atmosphere, particularly in an enclosed or substantially enclosed growth environment such as an “incubation chamber” or “growth chamber.” In some aspects, the present disclosure provides for a growth environment having a growth atmosphere that is maintained during the incubation time period by replenishing the growth environment with one or more of the atmospheric gases, such as CO₂, replenishing the growth environment with air having the same composition as the target growth atmosphere composition, venting the growth environment to reduce content of one or more gases, or a combination thereof. In a non-limiting example, if the CO₂ content in a growth chamber is below a target set point, CO₂ gas can be infused into the growth chamber. Conversely, if the CO₂ content exceeds a target set point, then fresh air having the target growth atmosphere composition can be introduced into the growth chamber while venting the chamber to release the existing air having the high CO₂ content. Accordingly, growth chamber atmospheric content can be maintained via CO₂ and fresh air infusion to maintain a target CO₂ set point; as such, O₂ and other atmospheric components are maintained indirectly and fluctuate as a function of fungal respiration. In some other aspects, the present disclosure provides for a growth environment wherein the growth atmosphere CO₂ and O₂ contents are allowed to modulate with fungal respiration, without adjusting the growth atmosphere to maintain preselected CO₂ or O₂ content. Thus, the growth environment can be a closed system. The present disclosure also provides for a growth environment wherein the growth atmosphere CO₂ and O₂ contents are allowed to modulate with fungal respiration, and further allowing for adjustments to be made to the growth atmosphere under conditions wherein a particular preselected growth atmospheric condition is breached. In a non-limiting example, an aerial mycelium (from extra-particle aerial mycelial growth) can be grown in a growth atmosphere that allows for natural fungal respiration to occur, with a preselected CO₂ content ranging from about 0.02% to about 7% CO₂ (v/v), wherein the CO₂ content is adjusted (e.g., by injection of CO₂ into the growth atmosphere) if the CO₂ content falls outside the scope of the preselected range.

A growth environment of the present disclosure can be further characterized as having an atmosphere having a pressure as would be readily understood by a person of ordinary skill in the art in the mycelial cultivation industry. In a non-limiting embodiment, a growth atmosphere of the present disclosure can have an atmospheric pressure within a range of about 27 to about 31 inches of mercury (Hg), can have an atmospheric pressure of about 29 to about 31 inches Hg, or can have an atmospheric pressure of about 29.9 inches Hg. In some embodiments, a growth environment of the present disclosure can be characterized as having an ambient atmospheric pressure.

In some aspects of the present disclosure, the growth environment suitable for the growth of the aerial mycelia (from extra-particle aerial mycelial growth) of the present disclosure is characterized as having an airflow. In some further aspects, the air composition of the airflow can be substantially the same as the composition of the growth environment atmosphere. In some embodiments, an airflow can be used to direct and/or deposit aqueous mist that is present in the growth environment towards or onto a growth matrix (including nutritive substrate) and/or growing mycelium. The skilled person can adopt various means of directing the flows of air, including baffles, perforated barriers, airflow boxes and/or other tools that can be suitably positioned in the growth environment or in relation to tools (or beds) containing growth matrix (including nutritive substrate) in order to achieve the desired outcome, including a somewhat or substantially homogeneous airflow, with respect to direction and/or velocity, across a plurality of growth matrices (including nutritive substrate(s)) in the growth environment, and/or a somewhat or substantially homogeneous introduction and/or deposition of mist in the growth environment.

“Horizontal airflow” as used herein refers to flows of air directed substantially parallel to the surface of a growth matrix (including nutritive substrate) and any subsequent extra-particle mycelial growth (aerial or otherwise).

In some other aspects, the method of preparing an aerial mycelium (from extra-particle aerial mycelial growth) of the present disclosure can include directing an airflow through the growth environment. In some embodiments, the airflow can be a relatively high airflow environment, wherein the airflow can have a velocity of greater than about 250 linear feet per minute (lfm). In other embodiments, the airflow can be a relatively lower airflow environment, wherein the airflow can have a velocity of less than about 150 lfm, less than about 125 lfm, less than about 100 lfm, or less than about 75 lfm. In some more particular embodiments, the growth environment can have an airflow, wherein the airflow velocity is less than about 50 lfm, less than about 40 lfm, less than about 30 lfm, or less than about 25 lfm.

In some embodiments, the airflow is a substantially horizontal airflow. In some embodiments, the substantially horizontal air flow can have a velocity of no greater than about 350 lfm, or a velocity no greater than about 300 lfm. In other embodiments, the substantially horizontal airflow can have a velocity of no greater than about 275 lfm, a velocity of no greater than about 175 lfm, a velocity of no greater than about 150 lfm, a velocity of no greater than about 125 lfm, or a velocity of no greater than about 110 lfm. In some further embodiments, the velocity is at least about 5 lfm, at least about 10 lfm, at least about 15 lfm, at least about 20 lfm, at least about 25 lfm, at least about 30 lfm, at least about 35 lfm, at least about 40 lfm, at least about 45 lfm or at least about 50 lfm. In some more particular embodiments, the substantially horizontal airflow has mean velocity of about 5 lfm, about 10 lfm, about 15 lfm, about 20 lfm, about 25 lfm, about 30 lfm, about 35 lfm, about 40 lfm, about 45 lfm, about 50 lfm, about 55 lfm, about 60 lfm, about 65 lfm, about 70 lfm, about 75 lfm, about 80 lfm, about 85 lfm, about 90 lfm, about 95 lfm, about 100 lfm, about 105 lfm, about 110 lfm, about 115 lfm, or about 120 lfm. In some more particular embodiments still, the substantially horizontal air flow can have a velocity within a range of about 5 lfm to about 125 lfm, within a range of about 5 lfm to about 100 lfm, within a range of about 5 lfm to about 75 lfm, or within a range of about 5 lfm to about 50 lfm. In yet more particular embodiments, the substantially horizontal air flow can have a velocity within a range of about 5 lfm to about 40 lfm, or within a range of about 5 to about 25 lfm. In other embodiments, the substantially horizontal air flow can have a velocity within a range of about 40 lfm to about 120 lfm. Without being bound to any particular theory, the flows of air can facilitate the distribution of mist throughout the growth environment, can facilitate the distribution of mist onto the growth matrix (including nutritive substrate) surface and/or extra-particle mycelial growth (such as aerial), or both. The air flow and misting methods and associated apparatus, can be tuned in concert to achieve the desired mist deposition rate and/or mean mist deposition rate, and to tune the mycelial tissue morphology.

In some embodiments, aerial mycelia (from extra-particle aerial mycelial growth) can be prepared by exposing a growth matrix to aqueous mist throughout a portion of the incubation time period (e.g., by introducing mist into the growth environment throughout a portion of the incubation time period). Applicant has measured vertical expansion kinetics of mycelia over the course of an entire incubation period and has characterized the kinetics as having a primary myceliation phase and a vertical expansion phase. The primary myceliation phase included days 1 to 3 of the incubation time period. Introducing aqueous mist throughout a portion of the incubation time period (wherein the portion included the vertical expansion phase), and not introducing aqueous mist on days 1 to 3 of the incubation time period was sufficient to produce aerial mycelium (from extra-particle aerial mycelial growth) having substantially similar characteristics to aerial mycelia (from extra-particle aerial mycelial growth) obtained by depositing mist throughout the entire incubation period.

The desired airborne mist concentration value, and/or the control of the airborne mist concentration level in response to the mist concentration value, for improved growth may be different during different phases of the growing cycle (including zero). Further, the desired airborne mist concentration value, and/or the control of the airborne mist concentration level in response to the mist concentration value, for improved growth may also be different based on the organism generating the aerial mycelia (from extra-particle aerial mycelial growth). Some aspects of the present disclosure provide for a method of growing an aerial mycelium (from extra-particle aerial mycelial growth) comprising exposing a growth matrix (including nutritive substrate) to a growth environment comprising aqueous mist throughout the incubation time period (e.g., by introducing aqueous mist into the growth environment throughout the incubation time period, i.e., throughout the entire incubation time period). In other aspects, the present disclosure provides for a method of making an aerial mycelium (from extra-particle aerial mycelial growth) comprising exposing a growth matrix (including nutritive substrate) to aqueous mist throughout a portion of the incubation time period (e.g., by introducing aqueous mist into the growth environment throughout a portion of the incubation time period). In some embodiments, a portion of the incubation time period can comprise a vertical expansion phase. In some further embodiments, a portion of the incubation time period can further comprise at least a portion of a primary myceliation phase. In some other embodiments, a portion of the incubation time period can exclude a primary myceliation phase. In yet some other embodiments, a portion of the incubation time period can comprise a vertical expansion phase. Accordingly, in some aspects, introducing aqueous mist into a growth environment throughout a portion of an incubation time period can comprise introducing aqueous mist into the growth environment throughout a vertical expansion phase. In some embodiments, introducing aqueous mist into the growth environment throughout a portion of the incubation time period can comprise introducing aqueous mist into the growth environment throughout a vertical expansion phase and can exclude introducing aqueous mist during the primary myceliation phase. In some embodiments, the portion of the incubation time period can terminate at the end of a vertical expansion phase or can terminate at the end of an incubation time period.

In some other aspects, a portion of an incubation time period can begin during a first day, a second day, a third day or a fourth day of the incubation time period. Accordingly, in some aspects, introducing aqueous mist into a growth environment throughout a portion of an incubation time period can comprise introducing aqueous mist into the growth environment during a first, a second, a third or a fourth day of the incubation time period. In some embodiments, the portion of the incubation time period can terminate at the end of a vertical expansion phase or can terminate at the end of an incubation time period.

“Dry mass (DM) yield” as used herein refers to the bone-dry mass yield of aerial mycelium (from extra-particle aerial mycelial growth) from a standard mass of solid nutritive substrate. This is representative of the bioefficiency of the organism in converting the solid-nutritive substrate components into harvestable aerial mycelium.

### Discussion of Further Aspects of the Disclosure

The following discussion presents detailed illustrative descriptions of several embodiments of a method for preparing mycelium sheets and panels for further processing, and the mycelium sheets and panels produced therefrom. These embodiments and supporting examples are not intended to be limiting, and modifications, variations, combinations, etc., are possible and contemplated to be within the scope of this disclosure.

In a first embodiment of the disclosure, a method for preparing a mycelium sheet or panel material for further processing includes the steps of: growing a mycelium sheet or panel material including hyphae in a growth environment and upon a nutritive substrate, with the growing mycelium sheet or panel material including an original upper surface, which is the surface farthest from the nutritive substrate. The nutritive substrate is contained on a tool, and in one embodiment, the tool is selected from the group consisting of either a tray or bed. While the growing mycelium sheet or panel material is grown on the nutritive substrate, an upper portion of the growing mycelium sheet or panel material is removed, thereby creating a secondary upper surface of the growing mycelium sheet or panel material, which was beneath the original upper surface of the growing mycelium sheet or panel material. Essentially, the secondary upper surface is at an elevation that is lower than the original upper surface of the growing mycelium sheet or panel material. Following this removal of the upper surface mycelium material, the mycelium is further processed, such as for example, by being harvested off of the nutritive substrate, compressed, such as by being passed through a series of rolls or pressure plates, or treated with desired chemistry or liquids. In an alternative embodiment, the harvested mycelium sheet or panel material that has been separated from the nutritive substrate, is subject to a further rinsing step prior to textile processing. It has been theorized that a set of metabolites (growth chemistry in the youngest hyphae ends) is responsible for irregularities in color, texture, and surface composition, that may appear not only in recently harvested mycelium sheets and panels, but also in processed mycelium sheets and panels that are subjected to traditional textile manufacturing steps, such as compression and chemical treatments. By removal of such growth metabolites, it is theorized that such irregularities can be lessened or eliminated in their entirety. For the purposes of this disclosure, the term “primary metabolites” shall reference the metabolites most commonly present at the hyphal tips of mycelium on the original surface of a growing or grown mycelial sheet or panel material. The term “secondary metabolites” shall reference metabolites that may be present in a growing or grown mycelium sheet or panel material, such as at a location beneath the original surface of the growing or grown mycelium sheet or panel material, for example, at a location along the mycelium sheet or panel material thickness closer to the nutritive substrate than that of the original upper surface.

In one embodiment, the removal of the upper portion of the mycelium sheet or panel material is accomplished by a horizontal bandsaw which cuts through the sheet or panel material and thereby removes an upper portion of the growing mycelial sheet or panel material, prior to separating the remainder of the sheet or panel material from the nutritive substrate. In one alternative embodiment, a horizontal bandsaw can remove from about 0.5 cm and 3 cm of mycelial tissue from the upper portion of a mycelium sheet or panel material. In another alternative embodiment, a horizontal bandsaw can remove from about 1 cm and 1.5 cm of mycelial tissue from the upper portion of a mycelium sheet or panel material. For instance, a horizontal bandsaw can remove about 0.5 cm or more, such as about 0.6 cm or more, such as about 0.7 cm or more, such as about 0.8 cm or more, such as about 0.9 cm or more, such as about 1 cm or more of mycelial tissue from the upper portion of a mycelium sheet or panel material. The horizontal bandsaw can remove about 3 cm or less, such as about 2.8 cm or less, such as about 2.6 cm or less, such as about 2.4 cm or less, such as about 2.2 cm or less, such as about 2 cm or less, such as about 1.9 cm or less, such as about 1.8 cm or less, such as about 1.7 cm or less, such as about 1.6 cm or less, such as about 1.5 cm or less of mycelial tissue from the upper portion of a mycelium sheet or panel material.

In yet another alternative embodiment, a horizontal bandsaw can remove from about 5% and 50% of mycelial tissue from the upper portion of a mycelium sheet or panel material. In yet another alternative embodiment, a horizontal bandsaw can remove from about 10% and 25% of mycelial tissue from the upper portion of a mycelium sheet or panel material. For instance, a horizontal bandsaw can remove about 5% or more, such as about 6% or more, such as about 7% or more, such as about 8% or more, such as about 9% or more, such as about 10% or more, such as about 11% or more, such as about 12% or more, such as about 13% or more, such as about 14% or more, such as about 15% or more, such as about 16% or more, such as about 17% or more, such as about 18% or more, such as about 19% or more, such as about 20% or more of mycelial tissue from the upper portion of a mycelium sheet or panel material. The horizontal bandsaw can remove about 50% or less, such as about 45% or less, such as about 40% or less, such as about 35% or less, such as about 33% or less, such as about 31% or less, such as about 30% or less, such as about 29% or less, such as about 28% or less, such as about 27% or less, such as about 26% or less, such as about 25% or less, such as about 24% or less, such as about 23% or less, such as about 22% or less, such as about 21% or less, such as about 20% or less of mycelial tissue from the upper portion of a mycelium sheet or panel material.

In still another alternative embodiment, a horizontal bandsaw can remove an upper portion of a mycelial sheet or panel material such that the remaining base of the mycelial sheet or panel material is from about 1 cm and 10 cm tall. In yet another alternative embodiment, a horizontal bandsaw can remove an upper portion of a mycelial sheet or panel material such that the remaining base of the mycelial sheet or panel material is from about 4 cm and 6 cm tall. For instance, a horizontal bandsaw can remove an upper portion of a mycelial sheet or panel material such that the remaining base of the mycelial sheet or panel material is about 1 cm or more, such as about 2 cm or more, such as about 3 cm or more, such as about 4 cm or more tall. The horizontal bandsaw can remove an upper portion of a mycelial sheet or panel material such that the remaining base of the mycelial sheet or panel material is about 10 cm or less, such as about 9 cm or less, such as about 8 cm or less, such as about 7 cm or less, such as about 6 cm or less tall.

In each of the instances, the removed portion includes the original upper surface of the growing mycelium sheet or panel material, such as the extra-particle aerial mycelium (which will ultimately lead to harvested aerial mycelium).

It should be understood that the percentages of removal are with respect to the overall height of the growing or grown mycelium having the original upper surface. For instance, if the growing mycelial panel has a height of 10 cm with an exposed original upper surface, a 20% removal of the upper portion would result in approximately an 8 cm height mycelial panel with a new upper surface (or secondary upper surface, which was originally beneath the original upper surface before the removal by cutting).

In a further embodiment, the removal of an upper portion of the mycelial sheet or panel material is accomplished by a previously inserted perforated layer, such as a net, scrim-like material, or mesh, which had been previously inserted on the growing extra-particle aerial mycelium and which allowed additional mycelial hyphae growth through the perforated layer. At a desired time within the mycelial growth cycle, the inserted perforated layer is then lifted away from a base of mycelial growth (mass of grown mycelium), that is still situated upon the nutritive substrate. This alternative upper portion removal may remove the upper section of the mycelial sheet or panel material from a base of mycelial growth, prior to the base growth being separated from the nutritive substrate. For instance, in one embodiment of such a method, once the growing mycelium has achieved a sufficient or desired height above the nutritive substrate (or layer covering the nutritive substrate), a perforated layer is placed on the growing mycelial tissue. All subsequent growth of mycelial hyphae passes through the perforated layer and is present above the perforated layer. This subsequent growth has a relative height or elevation higher than that of the perforated layer, with respect to the nutritive substrate upper surface (or layer upon the nutritive substrate upper surface). At the desired time of harvest, the perforated layer is delaminated from the lower mycelial mass (the mycelial mass making up the base upon the nutritive substrate), which in turn removes an upper portion of the growing or grown mycelial sheet or panel material. The base mycelial mass thereafter includes a new or secondary upper surface (which was below the original upper surface before delamination).

In one embodiment, the perforated layer for delaminating the upper portion of the growing mycelium is a net that is placed on the growing mycelium sheet or panel material. In an alternative embodiment, the perforated layer is placed on the growing mycelium from about 5 days and 8 days into the mycelium growth cycle. In another alternative embodiment, the perforated layer is placed on the growing mycelium from about 6 days and 7 days into the mycelium growth cycle.

In yet another alternative embodiment, the perforated layer is placed on the growing mycelium once the mycelium sheet or panel material has achieved a height of from about 3 cm and 9 cm. In still another alternative embodiment, the perforated layer is placed on the growing mycelium once the sheet or panel material has achieved a height of from about 5 cm and 7 cm. For instance, the perforated layer is placed on the growing mycelium once the sheet or panel material has achieved a height of about 3 cm or more, such as about 4 cm or more, such as about 5 cm or more. The perforated layer is placed on the growing mycelium once the sheet or panel material has achieved a height of about 9 cm or less, such as about 8 cm or less, such as about 6 cm or less, such as about 5 cm or less.

In yet another alternative embodiment, the perforated layer is placed on the growing mycelium from about 50% and 95% of the way through the mycelium growth cycle. In another alternative embodiment, the perforated layer is placed on the growing mycelium from about 75% and 90% of the way through the mycelium growth cycle. For instance, the perforated layer is placed on the growing mycelium about 50% or more, such as about 60% or more, such as about 65% or more, such as about 70% or more, such as about 75% or more of the way through the mycelium growth cycle. The perforated layer is placed on the growing mycelium about 95% or less, such as about 90% or less, such as about 85% or less, such as about 80% or less, such as about 75% or less of the way through the mycelium growth cycle.

Once the upper portion of the mycelium is removed, such as by cutting or delaminating the upper portion of the extra-particle aerial mycelium from the base layer of extra-particle aerial mycelium (which is still attached to the nutritive substrate), the remaining base layer comprising the sheet or panel material may be harvested from the nutritive substrate. The secondary upper surface from which the upper portion was removed may still be covered by secondary metabolites that will darken the color of the harvested sheet or panel material in later processes, if they are not removed before conducting such later processing.

It therefore may be further useful to conduct a second metabolite reduction step on the mycelium material prior to or in conjunction with textile manufacturing steps. For instance, in still another embodiment of the method, a second metabolite-reduction step is included in the overall process (in addition to the physical removal step of removal of primary metabolites in the upper mycelium sheet or panel material portions, that is achieved by the cutting or delaminating actions, for example). The second metabolite-reduction step may be accomplished on the remaining base mass of mycelium sheet or panel material by a rinsing action, conducted on the harvested mycelium material after its removal (physical separation) from the nutritive substrate (whether it be a liquid or solid-state nutritive substrate).

In one alternative embodiment, the harvested mycelium sheet or panel material is rinsed in water for from about 5 seconds and 60 minutes. In another alternative embodiment, the harvested mycelium sheet or panel material is rinsed in water for from about 10 seconds and 10 minutes. In yet another alternative embodiment, the harvested mycelium sheet or panel material is boiled in water for from about 6 minutes and 60 minutes. In still another alternative embodiment, the harvested mycelium sheet or panel material is boiled in water for from about 5 seconds and 20 seconds. In yet another alternative embodiment of the disclosure, the type of water used in the rinsing step can include one or more of tap water, filtered water, reverse osmosis water, distilled water, deionized water, or softened water. In still another alternative embodiment of the disclosure, the conductivity of the water in a rinsing step can exist in a range of from about 0.5 and 1500 uS/cm (microsiemens per centimeter). In still a further alternative embodiment, the temperature of the water in a rinsing step can range from about 1° C. and 100° C. In another alternative embodiment of the disclosure, the temperature of the water in a rinsing step can range from about 20° C. and 30° C.

In still another alternative embodiment of the disclosure, the harvested mycelium sheet or panel material is rinsed in an acid solution for from about 5 seconds and 60 minutes. In another alternative embodiment of the disclosure, the harvested mycelium sheet or panel material is rinsed in an acid solution for from about 10 seconds and 10 minutes. In still another alternative embodiment of the disclosure, the harvested mycelium sheet or panel material is rinsed by being boiled in an acid solution for from 6 minutes and 60 minutes. In another alternative embodiment of the disclosure, the harvested mycelium sheet or panel material is rinsed by being boiled in an acid solution for from about 5 seconds and 20 seconds.

In another alternative embodiment of the disclosure, the acid molecule used to make the rinsing solution for a rinsing step of a harvested mycelium sheet or panel material can include an acid selected from the group consisting of acetic acid, formic acid, nitric acid, phosphoric acid, citric acid, sulfuric acid, and hydrochloric acid. In still another alternative embodiment of the disclosure, the concentration of an acid in such a rinsing solution can range from about 0.1%-10%, alternatively, from about 0.5% and 5%. In still another alternative embodiment of the disclosure, the temperature of the acid rinsing solution can range from about 1° C.-100° C. In still another alternative embodiment of the disclosure, the temperature of the acid rinsing solution can range from about 20° C.-30° C.

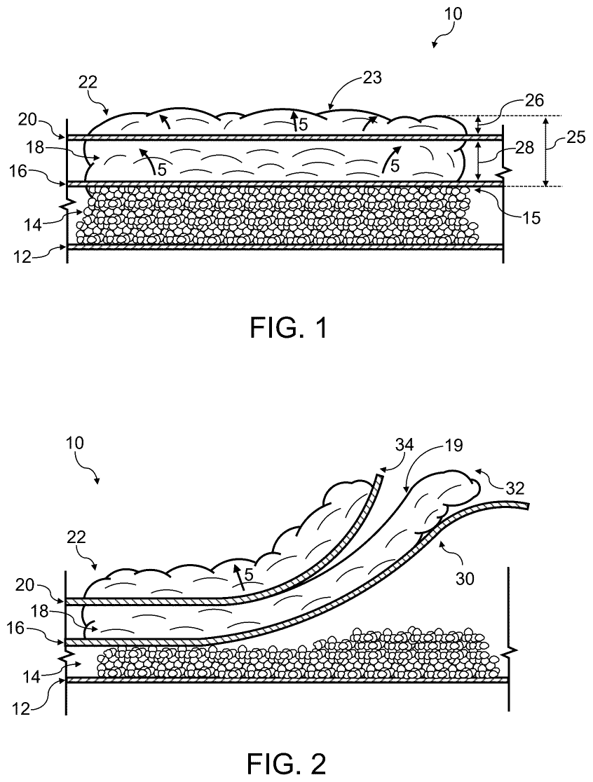

With specific reference to the figures, as can be seen in FIG. 1, a partial cross-sectional view of a growing mycelium panel is illustrated as 10. The growing mycelium panel 10 is growing in an unshown growth environment and is situated on a solid particulate nutritive substrate 14 having a nutritive substrate upper surface 15, which is itself situated on a bed 12. Alternatively, the nutritive substrate 14 may be situated in a tray (not shown). In this particular embodiment, a perforated layer 16 is situated on the nutritive substrate 14. Such perforated layer may be used to provide additional physical attributes to the grown mycelium panel, or alternatively, may be used to assist in the removal of the mycelium panel during harvest. Such perforated layer may stay with the nutritive substrate or alternatively be removed with the mycelium panel. Mycelium hyphae which grow from a growth matrix including the nutritive substrate 14 extend through the perforated layer 16, such as a scrim or mesh material, to the exposed atmosphere in the growth environment (and the airborne mist present in such growth environment). The general negatively gravitropic direction of growth upward 5 is illustrated by directional arrows. As the bulbous mycelial tissue 18 grows upward and away from the nutritive substrate (forming a three-dimensional panel of relatively significant thickness, and which layer makes up the base layer of the final mycelium panel), a second perforated layer 20 is positioned on the growing extra-particle aerial mycelium. The growing extra-particle aerial mycelium (shown as the bulbous growths 18 of the base layer) extend through the second perforated layer 20 (which in one embodiment is a net) to form an upper portion 22 to the mycelium panel, having an original upper surface 23. The total thickness of the mycelium panel is illustrated as 25, with a base layer of the mycelium panel illustrated as having thickness 28, and an upper portion of the panel illustrated as having thickness 26.

As can be seen in the partial cross-sectional view of FIG. 2, the grown mycelium panel of FIG. 1 is being removed. Specifically, the upper portion of the mycelium panel 22 is being delaminated by the raising of the second perforated layer 20 off of the underlying base layer of the mycelium panel 18, having a secondary upper surface 19. Such secondary upper surface 19 is relatively smooth compared with the topography of the bulbous upper portion 22, original upper surface 23. The movement of delamination 34 is illustrated, lifting the upper portion 22 away from the underlying base layer 18 of the mycelium panel. Concurrently or separately, the base layer 18 of the mycelium panel may be lifted up and away 32 off of the first perforated layer 16 (which separates the mycelium panel from the underlying nutritive substrate 14) and which may be lifted up and away from the nutritive substrate 14. As noted, the first perforated layer 16 may become part of the final mycelium panel in order to provide desirable physical attributes to the grown mycelium panel (such as strength for example). It should be recognized that while two perforated layers are illustrated in the embodiment of FIGS. 1 and 2, it is contemplated that none or one perforated layer be utilized. For instance, the first perforated layer may be left out, and instead the base layer of the mycelium panel may be cut off of the nutritive substrate by a cutting mechanism, such as a horizontal bandsaw. Alternatively, the second perforated layer may be left out, and the upper portion may be cut off of the base layer of the mycelium panel with a horizontal bandsaw.

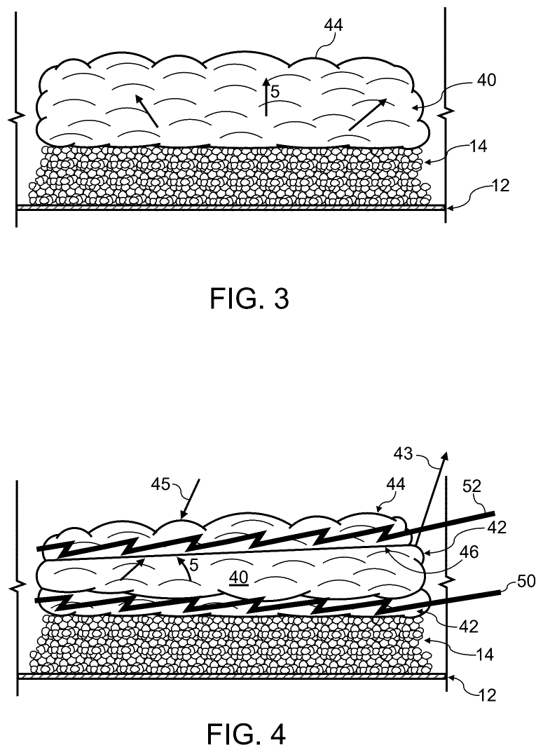

An alternative embodiment of the method of preparing a grown mycelium panel (and/or sheet) for further processing is illustrated in the partial cross-sectional views of FIGS. 3 and 4. As can be seen in the figures, instead of using one or more embedded perforated layers in a growing mycelium mass, the growing mycelium mass 40 is allowed to grow as a continuous panel upon nutritive substrate 14 without the use of embedded perforated layers. Instead of using perforated layers to assist in separating/removing portions of the mycelium panel from the nutritive substrate below 14 (which is itself situated on a tool 12 such as a bed), a series of horizontal bandsaws 50, 52 are used to cut away portions of the mycelium panel. For example, a horizontal bandsaw 52 is used to cut away an upper portion 44 of the mycelium panel from a base layer 42 of the mycelium panel in the direction 43 away from the base layer 42. The upper portion 44 includes an original upper surface 45. The separated base layer 42 of the mycelium panel has a secondary upper surface 46. A horizontal bandsaw 50 is also used to cut or harvest the base layer of the mycelium panel 42 off of the nutritive substrate 14, leaving a relatively thin portion of the base layer 47 attached to the nutritive substrate 14. It should be noted, that while in FIG. 4, two cutting mechanisms (bandsaws) are illustrated separating and effectuating the removal of the various layers of mycelium panel from one another and also from the nutritive substrate, various techniques may be utilized to carry out the layer separation. For instance, a cutting method may be used to separate the base layer of the mycelium panel from the nutritive substrate, while an embedded perforated layer may be used to separate (and remove) an upper portion of the growing mycelium panel from a lower base layer of the growing mycelium panel.

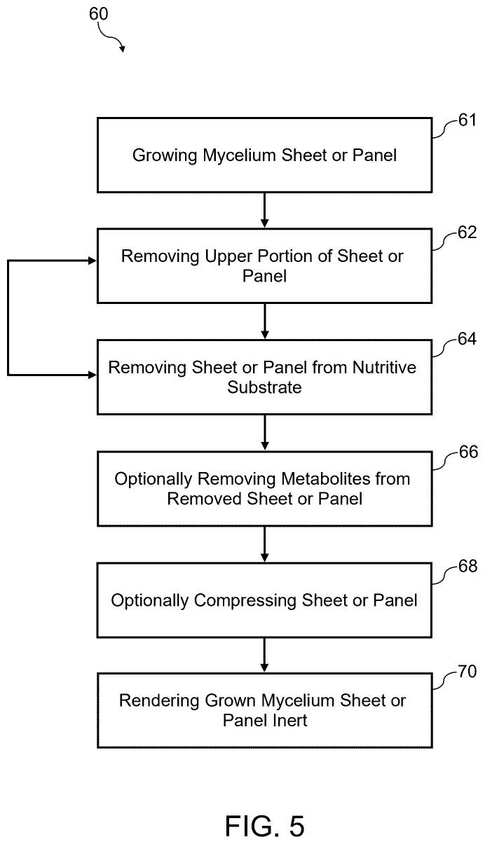

A flow/process diagram of an exemplary method for preparing a grown mycelium sheet or panel material for further processing 60 is illustrated in FIG. 5. The process 60 includes the steps of growing a mycelium sheet or panel 61 (in a suitable growth environment, and upon a nutritive substrate). Upon growth of the mycelium sheet or panel material, such sheet or panel material may first have an upper portion of its mass removed while its base layer is situated on a nutritive substrate, or alternatively, it may be separated from the supporting nutritive substrate, and then have its upper portion removed. Following removal of an upper portion of the mycelium sheet or panel material, the mycelium sheet or panel material may be removed or harvested from the nutritive substrate. Optionally, such removed or harvested mycelium sheet or panel material (actually the base layer of the sheet or panel material, the upper portion having already been removed) may then be subjected to a second metabolite-removal step 66 (such as rinsing), the first metabolite-removal step having been the physical removal of a layer of material from the mycelial mass. The mycelium sheet or panel material may then be optionally compressed 68 and then rendered inert 70 for further processing as desired.

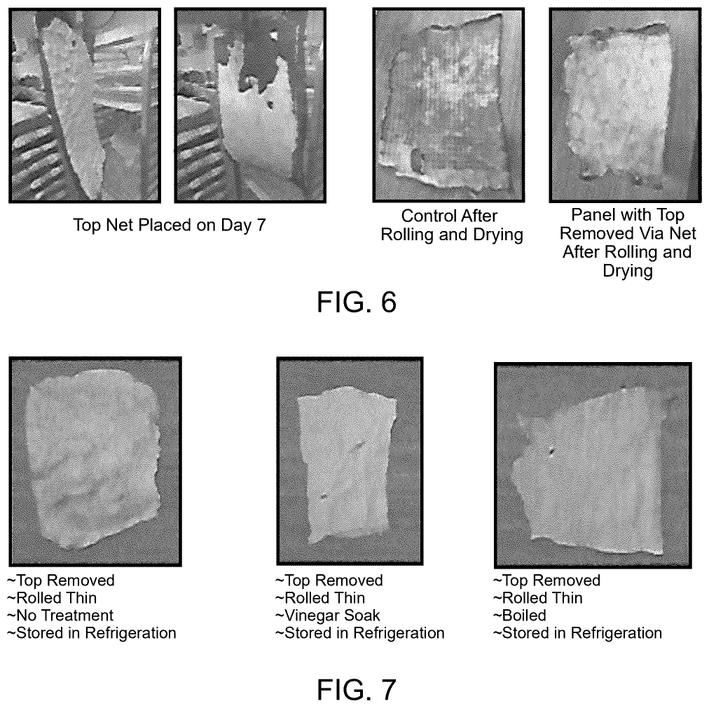

A series of comparative, exemplary photographs are presented as FIG. 6. FIG. 6 illustrates effect of removal of a top layer (upper portion) of a mycelium panel via a net device (left). The difference between a mycelium panel with the top section intact versus one in which the top section has been removed, post-drying (right) is evident. As can be seen in the figure, an upper perforated layer (aka a top net), has been placed on the growing mycelium panel at about day 7 in the mycelial growth cycle. The upper perforated layer (aka top net) was used to strip away an upper portion of the mycelium panel. The remaining base layer material was removed using the top net. A control sample without removal of an upper portion was also evaluated. Both samples were compressed using rolls and dried to render the material inert. The control sample, without upper portion removed, was noticeably browner in color. In contrast, a mycelium panel with the upper portion removed via a top net mechanism appeared lighter in color.

A series of exemplary, comparative photographs of various metabolite treatment results are illustrated in FIG. 7. As can be seen in the figure, images of panels with the upper portions removed compare the results of various secondary metabolite rinsing treatments with subsequent compression and drying steps.

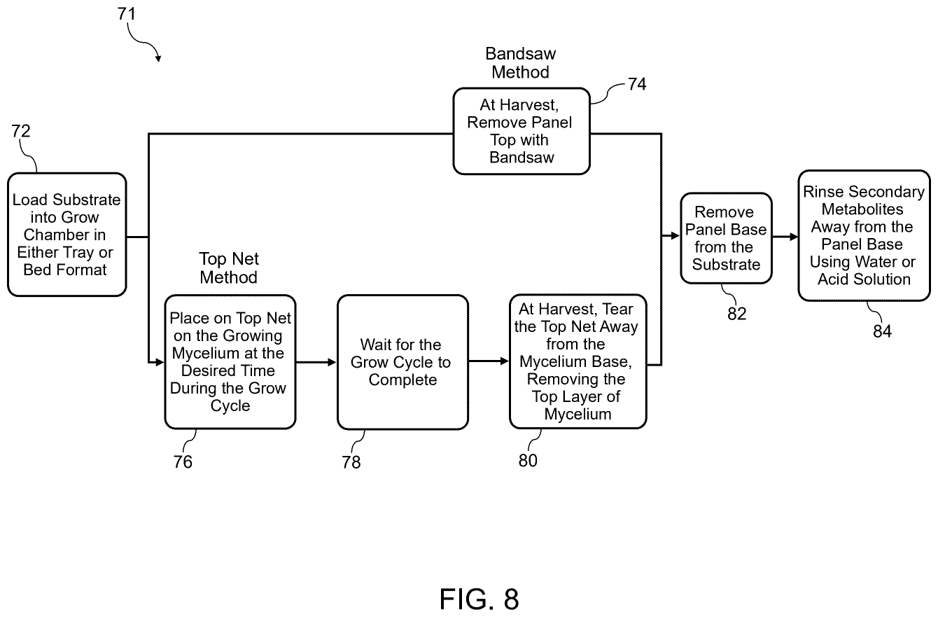

An exemplary process flow diagram for a method of preparing a grown mycelium sheet or panel material for further processing 71 is illustrated in FIG. 8. As can be seen in the figure, a nutritive substrate (identified as substrate in the figure) is loaded into a grow chamber (growth environment), such as in a tray or bed (such as a relatively continuous bed) format. If a cutting mechanism is to be used to separate an upper portion of the mycelium panel from a base layer, then at the time of harvest 74 (after the mycelium has grown to a desired height dimension) the desired thickness of a top layer (or upper portion) of the mycelium panel is removed with the use of the horizontal bandsaw. This physically removes the primary metabolites that are located at the youngest grown hyphae of the mycelium panel. Alternatively, if a perforated layer is to be used to effectuate separation of an upper portion (or top layer) of the mycelium panel from a lower base layer (such as through a top net) then a top net is placed on the growing mycelium panel at the desired time (or thickness) during the mycelium growth cycle 76. Such top net layer will essentially be embedded in the growing mycelium over time. A period of time is then allowed to elapse such that the growth cycle of the mycelium panel is completed (that is extra-particle aerial mycelial growth is completed in accordance with one embodiment) 78. Then at harvest, the upper portion (or top layer) is torn away (or delaminated) from the base layer of the mycelium panel by removal of the embedded perforated layer 80. This physically removes the primary metabolites that are located at the youngest grown hyphae of the mycelium panel. Following either the use of a cutting mechanism or an embedded perforated layer, the base layer of the mycelium panel is then removed from the nutritive substrate (either via an embedded layer or another cutting mechanism) 82. It should be recognized that while a bandsaw is described as a cutting mechanism of choice, other suitable cutting mechanisms are contemplated to be within the scope of the method, such as for instance a metal wire cutter. Following removal of the base layer of the mycelium panel from the nutritive substrate, the base layer of the mycelium panel may then be rinsed so that secondary metabolites may be removed from the base layer of the mycelium panel (such as by using a water or acid solution) 84.

This disclosure has described various embodiments of a method to improve the quality of grown mycelium-based sheet and panel materials, and its efficacy to post-harvest processing treatments in several ways. For instance, early removal of growing or grown mycelium material responsible for irregular or inconsistent physical and visual features (which may be immediately evident upon harvest or become evident at a later date), will prevent the mycelium-based sheet or panel material from producing a rigid and brittle exterior layer during or after final textile processing. Removal of growing mycelium material responsible for irregular or inconsistent physical and visual features, when performed on uncompressed mycelium material at or following harvest, allows for easier and more accurate removal of the less desirable material than when in the compressed form in subsequent textile-manufacturing processes. Removal of growing mycelial material responsible for irregular or inconsistent physical and visual features also produces a flatter top surface to mycelium sheet or panel materials, which may result in a reduction in the number and complexity of later textile processing steps. Finally, removal of growing mycelial material responsible for irregular or inconsistent physical and visual features early in the mycelium growth cycle, may allow for easier post-harvest chemistry application and more efficient dye uptake.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein but are to be accorded the widest scope consistent with this disclosure, the principles and the novel features disclosed herein.

While certain embodiments of the disclosure have been described, these embodiments have been presented by way of example only and are not intended to limit the scope of the disclosure. Indeed, the apparatus designs, methods, and systems incorporating such described herein, may be embodied in a variety of other forms. Furthermore, various omissions, substitutions and changes in the apparatus, methods, and systems described herein may be made without departing from the spirit of the disclosure. The accompanying claims and their equivalents are intended to cover such forms or modifications as would fall within the scope and spirit of the disclosure. Accordingly, the scope of the present disclosure is defined by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process, or system so disclosed.

The word “exemplary” is used herein to mean “serving as an example, instance, or illustration.” Any aspect or embodiment described herein as “exemplary” is not necessarily to be construed as preferred or advantageous over other aspects or embodiments. Various aspects of the novel systems, apparatus, and methods are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough, and will fully convey the scope of the disclosure to those skilled in the art. Based on the teachings herein one skilled in the art should appreciate that the scope of the disclosure is intended to cover any aspect of the systems, apparatus, and methods disclosed herein, whether implemented independently of, or combined with, any other aspect described. For example, an apparatus may be implemented, or a method may be practiced using any number of the aspects set forth herein. In addition, the scope of the disclosure is intended to cover such an apparatus, method or system which is practiced using other structure, functionality, or structure and functionality in addition to or other than the various aspects of the disclosures set forth herein. It should be understood that any aspect disclosed herein may be embodied by one or more elements of a claim.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Although features may be described above as acting in certain combinations, one or more features from a claimed combination can, in some cases, be excised from the combination, and the combination may be claimed as a subcombination or variation of a subcombination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z. Thus, as used herein, a phrase referring to “at least one of X, Y, and Z” is intended to cover: X, Y, Z, X and Y, X and Z, Y and Z, and X, Y and Z.

The headings provided herein, if any, are for convenience only and do not necessarily affect the scope or meaning of the devices and methods disclosed herein.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A method for preparing a mycelium sheet or panel material for further processing, the method comprising:
a) growing a mycelium sheet or panel material including hyphae in a growth environment and upon a nutritive substrate, with the growing mycelium sheet or panel material including an original upper surface,
b) removing an upper portion of the growing mycelium sheet or panel material, either prior to, or in conjunction with a mycelium sheet or panel material harvest, thereby exposing a secondary upper surface which had been located beneath the original upper surface, and
c) processing the growing mycelium sheet or panel material including the secondary upper surface into a mycelium-based textile product.

2. The method of claim 1, wherein the method further includes compressing the growing mycelium sheet or panel material.

3. The method of claim 2, wherein the compressing step is performed by one or more rollers or pressure plates.

4. The method of claim 1, wherein the method further includes rendering the growing mycelium sheet or panel material inert.

5. The method of claim 4, wherein the rendering is accomplished by exposure of the growing mycelium sheet or panel material to an action comprising heating, drying, or a combination thereof.

6. The method of claim 1, wherein the removing step is accomplished by inserting a perforated layer upon the growing mycelium sheet or panel material, whereby the growing mycelium sheet or panel material hyphae penetrate the perforated layer to create the upper portion of the growing mycelium sheet or panel material, and the perforated layer along with mycelium hyphae which have subsequently grown through the perforated layer are removed, thereby exposing a secondary upper surface of growing mycelium which had most recently not been the mycelium sheet or panel material original upper surface.

7. The method of claim 1, wherein the removing step is accomplished by a cutting mechanism.

8. The method of claim 7, wherein the removing step is accomplished by a bandsaw.

9. The method of claim 8, wherein the bandsaw is a horizontal bandsaw.

10. The method of claim 1, further including a rinsing step following the removing step.

11. The method of claim 1, further including a harvesting step, wherein the mycelium sheet or panel material is removed from the nutritive substrate.

12. The method of claim 11, wherein the rinsing step follows the harvesting step.

13. A method for preparing mycelium sheet or panel material for further textile processing, the method comprising:
a) growing a mycelium sheet or panel material including hyphae in a growth environment and upon a nutritive substrate, with the growing mycelium sheet or panel material including an original upper surface,
b) removing an upper portion of the growing mycelium sheet or panel material, thereby exposing a secondary upper surface on a base layer of the mycelium sheet or panel material, which secondary upper surface had been located beneath the original upper surface, and further, which base layer of the mycelium sheet or panel material still being situated upon the nutritive substrate,
c) harvesting the mycelium sheet or panel material from the nutritive substrate, and
d) processing the harvested growing mycelium sheet or panel material including the secondary upper surface into a mycelium-based textile product.

14. The method of claim 13, wherein the mycelium sheet or panel material includes aerial mycelium.

15. The method of claim 13, wherein the nutritive substrate comprises a liquid state nutritive substrate, a solid state nutritive substrate, or a combination thereof.

16. The method of claim 13, further including rinsing the harvested mycelium sheet or panel material.

17. A mycelium sheet or panel material produced by the method of claim 1.

18. A mycelium sheet or panel material produced by the method of claim 1, the mycelium sheet or panel material demonstrating a noticeable reduction in color than a comparable mycelium sheet or panel material not having an upper portion removed prior to further processing.

19. A mycelium sheet or panel material produced by the method of claim 1, the mycelium sheet or panel material demonstrating a noticeable reduction in surface irregularities than a comparable mycelium sheet or panel material not having an upper portion removed prior to further processing.

20. A mycelium sheet or panel material produced by the method of claim 1, the mycelium sheet or panel material demonstrating a noticeable reduction in brittleness than a comparable mycelium sheet or panel material not having an upper portion removed prior to processing.
